(19) United States
(12) Patent Application Publication
(10) Pub. No.: US 2012/0022015 A1
(43) Pub. Date: Jan. 26, 2012

(54) METHOD OF TREATMENT AND SUPPLEMENT

(76) Inventor: Philip George, Scarsdale, NY (US)

(21) Appl. No.: 12/841,780

(22) Filed: Jul. 22, 2010

(51) Int. Cl. A61K 31/714; A61P 35/00; A61P 7/06; A61P 25/28

(52) U.S. Cl. 514/52

(57) ABSTRACT

A method of treatment and associated nutritional combination is disclosed. The method of treatment includes providing a patient with a nutritional combination including appropriate amounts of riboflavin, pyridoxine hydrochloride, and cyanocobalamin. The combination may further include thiamine mononitrate and calcium pantothenate, and may be in the form of an oral supplement such as a capsule or tablet. Alternatively, the combination may be a fluid for intravenous intake, and may further include thiamine hydrochloride, D-panthenol, water, benzyl alcohol, and sodium chloride. The treatement and combination of ingredients works synergistically together and is particularly effective in enhancing nerve regeneration in a variety of patients.

### BACKGROUND OF THE INVENTION

1. Field of the Invention

The present invention relates to a method of treatment and supplement, and more particularly, a method of treating an individual with a nutritional supplement and a nutritional supplement.

2. Background

Many nutritional combinations and supplements have been proposed and given to patients for numerous reasons. The benefits of various vitamins and nutritional supplements have been studied and are known. However, in treating patients having various conditions, and particularly those in need of nerve generation, conventional treatments have not been sufficiently effective.

Among conventional nutritional combinations previously known, U.S. Pat. No. 6,998,501 issued to Wright et al., discloses a nutritional supplement for lowering serum triglyceride and cholesterol levels. While certain ingredients are disclosed, the supplement lacks various ingredients which, in combination, would produce a beneficial effect.

U.S. Pat. No. 6,139,872, issued to Walsh, discloses a vitamin product with various other ingredients. However, this combination is also not completely capable of enhancing benefits to a patient with various needs.

Various different types of patients, such as those in need of nerve regeneration, neuromuscular therapy treatment, ALS patients, Alzheimer's patients, breast cancer patients, elevated cholesterol patients, are treated in various manners. However, a nutritional combination and treatment with the same which would benefit such patients as described below, and which can provide a preventative effect against such conditions, is not previously known.

Thus, there remains a need for a treatment involving a nutritional combination which better enhances nerve regeneration and provides overall benefits not previously provided to various patients.

### SUMMARY

The present invention is a method of treatment and a nutritional combination which provides enhanced benefits to a variety of patients.

The method of the present invention includes treating a patient by providing a nutritional combination. Preferably, the combination is provided at least once per day. It may be provided in oral form, such as via a tablet or capsule. Although, any other suitable form for oral consumption is contemplated to be within the scope of the present invention.

The nutritional combination of the present invention includes, at the least, riboflavin, pyridoxine hydrochloride, and cyanocobalamin. If provided in oral form, the combination preferably further includes suitable amounts of thiamine mononitrate and calcium pantothenate.

In embodiments in which the nutritional combination of the present invention is in the form of a fluid, in addition to riboflavin, pyridoxine hydrochloride, and cyanocobalamin, it further includes suitable amounts of thiamine hydrochloride, D-panthenol, water, benzyl alcohol, and sodium chloride. Thus, it will be suitable for intravenous treatment such as via an IV or injection.

The treatment of the present invention, and the corresponding nutritional combination, has components which have a synergistic effect when taken in combination in appropriate amounts, as will be detailed below. It is particularly effective for enhancing nerve regeneration. This can be a great benefit to a variety of patients including neuromuscular therapy patients, ALS patients, Alzheimers disease patients, patients suffering from elevated cholesterol, and breast cancer patients. Furthermore, it can act as a preventative measure for these conditions.

Still other objects and advantages of the present invention will become readily apparent to those skilled in the art from the following detailed description, wherein it is shown and described only the preferred embodiments of the invention, simply by way of illustration of the best mode contemplated of carrying out the invention. As will be realized, the invention is capable of other and different embodiments and its several details are capable of modifications in various obvious respects, without departing from the invention. Accordingly, the drawings, wherein like reference numerals represent like features, and description are to be regarded as illustrative in nature and not as restrictive.

### BRIEF DESCRIPTION OF THE DRAWINGS

There are no drawings necessary in order to describe the present invention.

### DETAILED DESCRIPTION OF THE INVENTION

While this invention is susceptible of embodiments in many different forms, there are shown in the drawings and will herein be described in detail, preferred embodiments of the invention with the understanding that the present disclosure is to be considered as an exemplification of the principles of the invention and is not intended to limit the broad aspect of the invention to the embodiments illustrated.

The present invention is a method of treating a patient and a nutritional combination for such use. The method includes providing a patient with a nutritional combination. The combination includes riboflavin (vitamin B2), pyridoxine hydrochloride (vitamin B6), and cyanocobalamin (vitamin B12). The combination can be taken as a supplement orally, or, alternatively, it can be taken intravenously.

In various embodiments in which the combination is taken orally, it may be in tablet, capsule, or any other suitable form for oral treatment. Preferably, the oral supplement also includes thiamine mononitrate (vitamin B1) and calcium pantothenate (vitamin B5). This combination of ingredients is particularly effective in nerve regeneration and additional benefits, as further described below.

The treatment may include the steps of repeatedly providing the above combination to a patients, at least once daily, for a plurality of days if not indefinitely.

This treatment may be given to any patient in need of nerve regeneration, or in need of treatment for any of the conditions described above, or as a preventative for any of the conditions described above.

If taken orally, the suitable amounts of the above ingredients would be as follows, ideally. Thiamine mononitrate is provided in the amount of approximately 12 mg, riboflavin is provided in the amount of approximately 5 mg, pyridoxine hydrochloride is provided in the amount of approximately 1.5 mg, cyanocobalamin is provided in the amount of approximately 20 mcg, and calcium pantothenate is provided in the amount of approximately 40 mg. These amounts are believed to produce the most effective results, however, modifications in these amounts are also contemplated to be within the scope of the present invention.

In various embodiments in which the combination is to be given intravenously, it may be in the form of a fluid and, in addition to the riboflavin (vitamin B2), pyridoxine hydrochloride (vitamin B6), and cyanocobalamin (vitamin B12), it preferably contains thiamine hydrochloride, D-panthenol, water, benzyl alcohol, and sodium chloride. In this form, the combination may be given to the patient in the form of an IV or injection.

In embodiments in which the treatment is provided intravenously, the amounts of the ingredients provided are believed to be most effective as follows: thiamine hydrochloride is provided in the amount of approximately 120 mg, riboflavin is provided in the amount of approximately 1.2 mg, pyridoxine hydrochloride is provided in the amount of approximately 80 mg, cyanocobalamin is provided in the amount of approximately 1200 mcg, D-panthenol is provided in the amount of approximately 40 mg, benzyl alcohol is provided in the amount of approximately 1.5% by volume of the combination, and sodium chloride is provided in the amount of approximately 50 mg.

Various benefits of the above ingredients, alone and most suitably in combination, include the following. Thiamine mononitrate is needed for carbohydrate metabolism and proper neural activity and functioning. Body cells require this vitamin. It assists the body in converting carbohydrates into energy. The thiamine helps the body produce blood, metabolize carbohydrates, and produce hydrochloric acid for digestion. Thiamine also enhances blood circulation and optimizes cognitive activity and brain functions. It is needed to keep mucus membranes healthy and maintain normal functioning of the nervous system, heart, and muscles.

Without enough thiamine, a patient may suffer from loss of appetite, poor digestion, chronic constipation, weight loss, mental depression, nervous exhaustion, insomnia, muscular weakness, leg cramps, slowed heart rate, irritability, and defective hydrochloric acid. Digestive disorders can result as well. Thus, including this ingredient with the other ingredients is desirable.

Riboflavin aids in metabolism, respiratory proteins, fats, and carbohydrates. It helps maintain muscle tone in the gastrointestinal tract and promotes health of the nervous system, skin, hair, eyes, mouth, and liver. A deficiency in riboflavin can lead to slowed growth, fatigue, digestive problems, cracks and sores around the corners of the mouth, eye fatigue, swollen magenta tongue, soreness of the lips, mouth, and tongue, and sensitivity to light. Thus, including this ingredient with the other ingredients is desirable.

Calcium pantothenate helps prevent depression. It helps in steroid hormone production, adrenal gland production, and nervous system function. It can help decrease the side effects of other drugs or medications. It assists in adrenal gland and nervous system function, and normal growth and development of the body. Thus, including this ingredient with the other ingredients is desirable.

Pyridoxine aids in the formation and maintenance of the nervous system. Without proper levels of pyridoxine, neurological problems and skin disorders may appear. Thus, including this ingredient with the other ingredients is desirable.

Cyanocobalamin helps prevent the progression of neurological disorders. If levels of cyanocobalamin are too low over a prolonged period of time, permanent degenerative lesions of the spinal cord can result. Low levels of cyanocobalamin can also result in deadness of the hands and feet, loss of memory, poor coordination, insomnia, drowsiness, constipation, weight loss, nausea, and fatigue. However, supplementing cyanocobalamin can assist in maintaining a stronger nervous system, guarding against body fatigue and weakness, controlling cholesterol, and proper functioning of the heart. It can also help prevent the onset of sickle cell disorders, Alzheimer's disease, anemia, and breast cancer. Thus, including this ingredient with the other ingredients is desirable.

Absorption of thiamine, it must be understood, is dependent upon sufficient amounts of pyridoxine and cyanocobalamin being present. Thus, in order to be able to absorb B1 properly, the body must have sufficient B6 and B12. In combination, these ingredients aid in nerve regeneration in a synergistic fashion. Such a combination also has a preventative effect against ALS, Alzheimer's Disease, elevated cholesterol, and breast cancer.

While specific embodiments have been illustrated and described, numerous modifications come to mind without significantly departing from the spirit of the invention and the scope of protection is limited by the scope of the accompanying claims.

What is claimed is:

1. A method of treating a patient comprising the steps of:
providing a patient with a nutritional combination, wherein said combination comprises riboflavin, pyridoxine hydrochloride, and cyanocobalamin.

2. The method of treating a patient according to claim 1, wherein said combination further comprises thiamine mononitrate and calcium pantothenate.

3. The method of treating a patient according to claim 2, wherein said nutritional combination is provided to the patient in the form of an oral supplement.

4. The method of treating a patient according to claim 3, wherein said oral supplement is a tablet.

5. The method of treating a patient according to claim 3, wherein said oral supplement is a capsule.

6. The method of treating a patient according to claim 3, wherein said thiamine mononitrate is provided in the amount of approximately 12 mg, and wherein said riboflavin is provided in the amount of approximately 5 mg, and wherein said pyridoxine hydrochloride is provided in the amount of approximately 1.5 mg, and wherein said cyanocobalamin is provided in the amount of approximately 20 mcg, and wherein said calcium pantothenate is provided in the amount of approximately 40 mg.

7. The method of treating a patient according to claim 3, further comprising the steps of repeating the step of providing a patient with a nutritional combination at least once a day for a plurality of days to enhance nerve regeneration.

8. The method of treating a patient according to claim 3, further comprising the steps of repeating the step of providing a patient with a nutritional combination at least once a day for a plurality of days to enhance nerve regeneration for neuromuscular therapy.

9. The method of treating a patient according to claim 1, wherein said combination further comprises thiamine hydrochloride, D-panthenol, water, benzyl alcohol, and sodium chloride.

10. The method of treating a patient according to claim 9, wherein said combination is in the form of a fluid and is provided intravenously.

11. The method of treating a patient according to claim 10, wherein said thiamine hydrochloride is provided in the amount of approximately 120 mg, wherein said riboflavin is provided in the amount of approximately 1.2 mg, wherein said pyridoxine hydrochloride is provided in the amount of approximately 80 mg, wherein said cyanocobalamin is provided in the amount of approximately 1200 mcg, wherein said D-panthenol is provided in the amount of approximately 40 mg, wherein said benzyl alcohol is provided in the amount of approximately 1.5% by volume of said combination, and wherein said sodium chloride is provided in the amount of approximately 50 mg.

12. The method of treating a patient according to claim 10, further comprising the steps of repeating the step of providing a patient with a nutritional combination at least once a day for a plurality of days to enhance nerve regeneration.

13. The method of treating a patient according to claim 12, further comprising the steps of repeating the step of providing a patient with a nutritional combination at least once a day for a plurality of days to enhance nerve regeneration for neuromuscular therapy.

14. A nutritional combination for treating a patient comprising:
riboflavin,
pyridoxine hydrochloride, and
cyanocobalamin.

15. The nutritional combination for treating a patient according to claim 14, wherein said combination is an oral supplement, and wherein said combination further comprises thiamine mononitrate and calcium pantothenate.

16. The nutritional combination for treating a patient according to claim 15, wherein said thiamine mononitrate is provided in the amount of approximately 12 mg, and wherein said riboflavin is provided in the amount of approximately 5 mg, and wherein said pyridoxine hydrochloride is provided in the amount of approximately 1.5 mg, and wherein said cyanocobalamin is provided in the amount of approximately 20 mcg, and wherein said calcium pantothenate is provided in the amount of approximately 40 mg.

17. The nutritional combination for treating a patient according to claim 16, wherein said oral supplement is a tablet for oral intake.

18. The nutritional combination for treating a patient according to claim 16, wherein said oral supplement is a capsule for oral intake.

19. The nutritional combination for treating a patient according to claim 14, wherein said combination is a fluid for intravenous treatment, and wherein said combination further comprises thiamine hydrochloride, D-panthenol, water, benzyl alcohol, and sodium chloride.

20. The nutritional combination for treating a patient according to claim 19, wherein said thiamine hydrochloride is provided in the amount of approximately 120 mg, wherein said riboflavin is provided in the amount of approximately 1.2 mg, wherein said pyridoxine hydrochloride is provided in the amount of approximately 80 mg, wherein said cyanocobalamin is provided in the amount of approximately 1200 mcg, wherein said D-panthenol is provided in the amount of approximately 40 mg, wherein said benzyl alcohol is provided in the amount of approximately 1.5% by volume of said combination, and wherein said sodium chloride is provided in the amount of approximately 50 mg.
